(19) United States
(12) Patent Application Publication
(10) Pub. No.: US 2016/0089320 A1
(43) Pub. Date: Mar. 31, 2016

(54) Non-Synthetic Emulsion-Based Lipid Formulations and Methods of Use

(71) Applicant: Barrie TAN, Amherst, MA (US)

(72) Inventor: Barrie TAN, Amherst, MA (US)

(21) Appl. No.: 14/866,726

(22) Filed: Sep. 25, 2015

Related U.S. Application Data
(60) Provisional application No. 62/056,685, filed on Sep. 29, 2014.

(51) Int. Cl. A61K 8/49; A61K 8/60; A23L 1/30; A61Q 19/00; A23L 1/302; A61K 8/06; A61K 8/31

(52) U.S. Cl. CPC A61K 8/498; A61K 8/06; A61K 8/602; A61K 8/31; A61Q 19/00; A23L 1/302; A23L 1/30; A23V 2002/00

(57) ABSTRACT

Saponin (quillaja and yucca) and terpenoid (mono- and di-) botanicals are used for emulsion-based lipids to produce stable nanoparticles of the active nutrients or pharmaceuticals by reducing surfactant usage (less than 5%) and by reducing particle size (less than 600 nm). Non-synthetic emulsion-based formulations enhance bioavailability and mitigate safety concerns. This nanoemulsion technology is suitable for oil-in-water ingredients including vitamin E tocotrienols, CoQ10s, curcuma terpenoids, symmetrical carotenoids, phenolics, lipid-soluble vitamins, and lipid-soluble pharmaceuticals.

### CROSS-REFERENCE TO RELATED APPLICATIONS

This is a Non-Provisional application, which claims priority to U.S. Provisional Application No. 62/056,685, which was filed on Sep. 29, 2014; the contents of which are all herein incorporated by this reference in their entireties. All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### BACKGROUND OF THE INVENTION

An emulsion is a solution of a heterogeneous dispersion of oil-in-water or water-in-oil. An emulsion solution needs a lipid phase (e.g., an oil-soluble drug and glycerides), an aqueous phase (e.g., water, often buffered), an interfacial phase (e.g., emulsifier/surfactant, often of non-ionic or anionic types), and mechanical energy. Emulsion-based systems are needed to deliver lipid-soluble bioactives, such as, oil-soluble nutraceuticals and pharmaceuticals. For example, vitamins A, D, E, K and CoQ10s, omega-3s, carotenoids, phenolics and water-insoluble drugs need to be in an emulsion to maximize absorption. Methods of making an emulsion include low-energy spontaneous emulsification and high-energy high-pressure emulsification.

Use of low-pressure equipment results in emulsions of particle sizes of 1 μm to 10 μm. Applications include butter, margarine, yogurt, bulk oils, and oils for softgel delivery, where the oil or fat content is high and the viscosity tends to be high. Products have a “lipid feel”.

Oil-in-water emulsions may require high-pressure equipment, and result in particle sizes of 100 nm to 600 nm. Applications for oil-in-water emulsions include milk products, beverages, soups and dressings. The viscosity tends to be low, as these applications have a more “aqueous-feel” and have a low oil or fat content.

Both low-energy homogenization and high-energy homogenization (to produce oil-in-water) emulsions are known and reported. However, while much is known about these emulsion procedures, there are significant technological gaps that have not been explored nor answered.

First, many emulsifiers and surfactants are synthetics (petrochemically derived) and their utility has not been questioned. Surprisingly, non-synthetic (botanically derived) emulsifiers are not used to any extent in practice. It is generally assumed that synthetic emulsifiers are safe and do not interact negatively with the nutrient/drug being encapsulated. It is not generally appreciated that synthetic emulsifiers can be antagonistic or interfere with the activity/function of the nutrient/drug.

Second, emulsifiers and surfactants are used at high quantities such that the surfactants are 2%-20% of the oil. Surprisingly, attempts to keep the surfactant usage at low levels (1% or less) were not made to any extent in practice.

Third, it is generally known that a smaller particle size makes more stable emulsions, especially under high-energy pressure homogenization that generates 100 nm-600 nm particle sizes, but such attempts are rare and not applied to any extent in practice.

Fourth, the above emulsion procedures used synthetic emulsifiers and surfactants, as well as high quantities of these emulsifiers and surfactants. Many nutrients and drugs are lipid soluble and not very bio-accessible nor bio-available. Surprisingly, natural products can emulsify and provide surfactancy to the lipid-soluble nutrient at much lower amounts. However, the natural surfactant's ability to do so and to provide stable nanoemulsions has not been proven.

Concerted efforts to form functionally effective and stable emulsions—especially with botanicals—of lipid-soluble pharmaceuticals and nutrients was unavailable, and it is the goal of the disclosed compositions and methods to provide a means to formulate them.

A detailed description of low-energy and high-energy homogenization is given to describe their characteristics (advantages and disadvantages) and applications. Low-energy homogenization produces microemulsions with large droplets (1 μm-10 μm).

High-energy homogenization techniques have many benefits. The nanoemulsion will have small droplets (100 nm-600 nm), which enhance bioavailability. This method has a low surfactant-to-oil ratio, which provides a high bioactive nutrient or drug concentration (5%-40%). This technology could aid in food-science applications by reducing flavor or taste alteration (hence enhancing acceptance), reducing the amount of excipient (hence increasing safety), and reducing micelles (hence increasing emulsion stability).

There are many potential applications of high-energy homogenization techniques. They can be used in beverage products that are either clear (3-5% tocotrienols; e.g., 5-10 mg/dosage or serving), semi-clear (7-10% tocotrienols; e.g., 20-40 mg/dosage or serving), or opaque (15-20% tocotrienols; e.g., 50-125 mg/dosage or serving). Additionally, they can be used for medical products, such as, aspirator and aerosol products (e.g., asthma, bronchitis, lung and airway inflammation), and eye drops for corneal route (cornea, anterior chamber, lens, uveal tissues) of application (e.g., cataract, dry-eye, macular degeneration, retinopathy, Chlamydia), as well as, conjunctival route (conjunctiva, sclera, choroid, retinal pigment epithelial layer, neural retina) of application (macular degeneration, macular edema, and retinopathy). Also, they can be used for injectable delivery systems, including subcutaneous (SQ), intravenous (IV) and intramuscular (IM); and toiletry products (shampoos and conditioners, toners, body washes and soaps, douches).

Synthetic surfactants used in solubilization of lipid materials have become ubiquitous—if not essential—as an ingredient in many medicinal and food formulations.

Unfortunately, petrochemical-based surfactants have disadvantages. They are known to reduce (or mitigate) the effects of the nutrients or drugs and/or reduce their absorption. Additionally, there are concerns about toxicity and allergenicity, particularly in the pediatric and geriatric population for which such emulsification formulations may be suited. Until now, these synthetic surfactants have been thought to be inert or inactive excipients. This is not the case. Therefore, these petrochemical-based synthetic surfactants need to be used sparingly and should be the “last resort” of usage.

On the other hand, natural surfactants—those that are botanically derived—until now have not been considered or used in formulations. Specifically, the advantages of natural surfactants include being a renewable resource (e.g., non-exhaustible), ecological (i.e. more biodegradable with environmental and aquatic safety), safe (e.g., hypo-allergenic, non-toxic), and preferred by the public (e.g., mild, natural, eco-friendly).

Additionally, the utility of natural surfactants in different formulations have surprising characteristics and advantages, including decreased quantities of surfactant needed for a particular use, larger loading of active nutrient/drug, smaller particle size, and a more stable emulsion.

Commonly used synthetic surfactants have been shown to inhibit uptake of various nutrients or drugs dose-dependently (e.g., tocotrienol, vitamin A, cancer medications) by 2-4 fold. This renders the emulsified ingredients ineffective or compromised. Furthermore, synthetic surfactants are used at high concentrations, from 5% to 50% of the emulsion, and result in large particle sizes of 1-10 μm with zeta potential of −29 mV to +29 mV. This renders the emulsified ingredients unstable.

The disclosed compositions and methods are significant improvements because only naturally occurring non-synthetic surfactants are used for formulations to mitigate safety issues, to enhance effectiveness, and to produce nanoparticles that are bio-accessible and stable in foods. Surprisingly, the amount of botanical surfactant used is much lower, typically 2%-20% of the ingredient, such that the surfactant:ingredient ratio is 1:5 to 1:200. The surfactant in the finished product formulation is 0.1 to 1.0%. The encapsulated nutrient/drug in emulsions using botanical surfactants has a particle size of 100 nm-300 nm, which is 10 to 50 times smaller than the particle size of emulsions made using synthetic surfactants (1 μm—10 μm), and an acceptable zeta potential of less than −30 mV and more than +30 mV, well within the stability range. This renders the emulsified ingredients bio-accessible/bio-available and stable.

Common synthetic surfactants may be grouped broadly into five or more different categories. By way of example, synthetic surfactants can be, a] water-soluble surfactants (polyethylene glycol [PEG], propylene glycol, pyrrolidone, methylacetamide, methylsulfoxide), b] non-ionic surfactants (polysorbates, sorbitans, esterified PEGs, cremophors, labrasols), c] water-insoluble lipids (synthetic and structured triglycerides, especially C6-8 short-chained triglycerides), d] phospholipids (chemically and structurally altered), and e] cyclodextrins.

The disclosed compositions and methods do not use any of these common synthetic surfactants, but use botanically derived natural surfactants and surfactant aids (e.g., saponins, plant essential oils, alcohols, saccharides, triglycerides, terpenoids, biopolymers, and phospholipids) to effect emulsification of nutrients and drugs.

It is desirable to have a low viscosity in solution mixtures for homogenization so as to achieve smaller particle size emulsions. The disclosed compositions and methods use natural compounds, including terpenoids (e.g., limonene, geraniol, farnesol, geranylgeraniol), and alcohols (e.g., ethanol, glycerol), to reduce the homogenization viscosity. It is desirable to attain the lowest achievable viscosity in emulsion technology. This causes the emulsified ingredients to have the lowest possible particle sizes.

This emulsion technology may be applied to many lipid-soluble nutrients. For example, these lipid-soluble nutrients include CoQ10 (ubiquinone and ubiquinol), vitamin Es (tocopherols and tocotrienols), omega-3s (DHAs and EPAs), and polyphenols (resveratrol, EGCG, and quercetin), terpenoids (policosanols, xanthorrhizol, tumerones, curcumenes), carotenoids (astaxanthin, zeaxanthin, lycopene, and beta-carotene), and other lipid vitamins of A, D, and K.

Natural tocotrienol ingredients commonly come from palm or annatto (Table 1). Nanoemulsions of palm-based and annatto-based tocotrienol generated particle sizes of 210 nm to 280 nm, and one in the 1-10 μm range. All used synthetic surfactants/emulsifiers (last row). Only one nanoparticle formulation made with synthetic emulsifiers was stable (−41 mV). The last example (last column) provides an example of annatto tocotrienol converted to nanoparticles (115 nm) that are stable (−64 mV), using only natural surfactants (quillaja saponius).

TABLE 1
Tocotrienol Emulsion Formulation

| | | | | | Annatto |
|---|---|---|---|---|---|
| | | | | | (Disclosed |
| Source | Palm | Annatto | Palm | Annatto | composition)* |
| Particle Size | 1,000-10,000 | 210-230 | 210-240 | 285 | 115 |
| (nm) | | | | | |
| Zeta Pot | — | −25 | −41 | −14 to +4 | −64 |
| (mV)* | | | | | |
| Surfactants | Tween 80 | Cremophor & | Tween 80 & | PLGA & | Plant Extract |
| | & Labrasol | Labrasol | Poloxamer/88 | PVA | (Quillaja Saponins) |

*For emulsion to be stable and not form clusters or aggregates the zeta potential generally needs to be in the range of less than −30 mV and more than +30 mV [FIG. 1].

### DEFINITIONS

Essential oil—An extract that is not saponifiable (i.e. not fat/oil-based) and is produced by a plant. It often belongs to the terpene family of compounds. Limonene is one such example.

Low-energy homogenization—A low energy source is provided to blend active nutrients and an emulsifier to produce particle droplets of 1 μm-10 μm. These emulsions are usually stable.

High-energy homogenization—A high energy source is provided typically as a second phase after undergoing a phase of low-energy homogenization to produce particle droplets of 100 nm-600 nm. These nanoemulsions may be unstable and need to be stabilized.

Emulsion—An emulsion (e.g., oil-in-water) occurs when a lipid substance (e.g., nutrient or drug) plus an emulsifier (also called surfactant) are subjected to homogenization.

Co-Solvents—They are natural ingredients are added to a premix solution with the intention to reduce the emulsified solution viscosity. This may be desirable because viscosity is inversely proportional to particle size.

Zeta potentials—They are a measure of particle droplets or emulsion stability. The smaller the particle size (as in nanoparticles), the greater the need to prove the emulsion is stable to be reducible to practice. Stable particle size means zeta potentials are in the range of less than −30 mV and more than +30 mV. The range is discontinuous because electrostatic forces will clump up these particles, rendering the emulsion unstable, when the mV electrostatic forces are weak (positively or negatively).

mV—A unit of potential difference equal to one thousandth (10⁻³) of a volt. The zeta potential (in mV) is measured by electrophoretic mobility.

Nutrients—This is the bioactive ingredient to be turned into nanoparticles. The bioactive ingredient has to be an oil- or lipid-soluble material, which can be a pharmaceutical, a vitamin, a botanical compound, or a natural extract.

Macro-nutrients—Nutrients that, when dispersed in oil-in-water emulsions using low-energy homogenization, result in particle sizes of 1 μm to 10 μm.

Micro-nutrients—Nutrients that, when dispersed in oil-in-water emulsions using high-energy homogenization, result in particle sizes of 100 nm to 600 nm.

Phase separation—For an oil-in-water to remain stable in finished foods and beverages, the emulsion should stay in a permanent suspension for a period of time and temperature. When the oil-in-emulsion breaks up, the two layers separate. The oil may float to the top or cream at the top. Alternatively, excipients may precipitate and settle in the aqueous medium.

### BRIEF SUMMARY OF THE INVENTION

Botanical emulsifiers/surfactants (such as, saponins from quillaja and yucca) and co-solvent/viscosity reducers (e.g., terpenoids and alcohols) are added to lipophilic nutrients (or drugs) and subjected to high-pressure mixing. This produces emulsions of nanoparticles (100 nm-600 nm) that are stable (where zeta potentials are less than −30 mV or greater than +30 mV). The disclosed compositions and methods were illustrated with a lipid-soluble vitamin E tocotrienol nanoemulsion and applied to different beverages and subjected to different conditions. The disclosed compositions and methods are particularly suited for oil-soluble nutrients, such as, vitamin E (tocotrienols and tocopherols), CoQ10 (ubiquinol and ubiquinone), curcuma terpenoids (xanthorrhizol, tumerones, curcumenes, and curcumins), symmetrical carotenoids (astaxanthin, zeaxanthin, lycopene, and beta-carotene), omega-3s (DHA and EPA), phenolics (policosanols, resveratrol, EGCG, and quercetin), and other lipid-soluble vitamins (A, D, K).

### BRIEF DESCRIPTION OF THE FIGURES

FIG. 1 shows the zeta potential required to yield a stable emulsion.

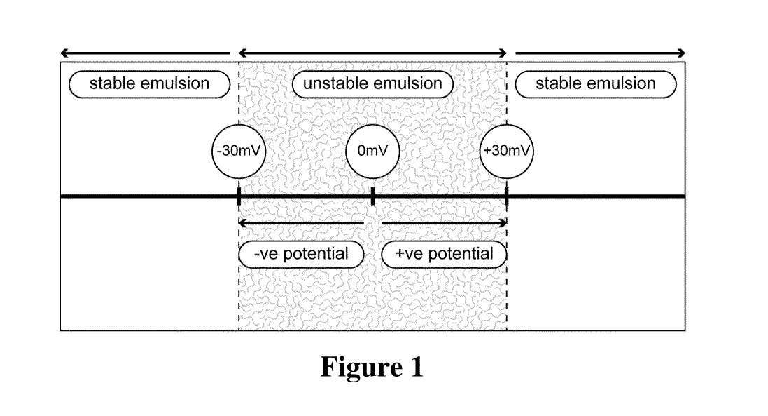

FIG. 2 shows the stability of botanically-derived tocotrienol nanoemulsions in lemonade at different times and storage temperature.

FIG. 3 shows the stability of botanically-derived tocotrienol nanoemulsions in chocolate (C) and milk (M) beverages. “+” means “with added nanoemulsion”.

FIG. 4 shows the stability of botanically-derived tocotrienol nanoemulsions in apple juice (A) and lemonade (L). “+” means “with added nanoemulsion”.

FIG. 5 shows the stability of botanically-derived tocotrienol nanoemulsions in water (W) and orange juice (O). “+” means “with added nanoemulsion”.

### DETAILED DESCRIPTION OF THE INVENTION

Emulsification is an important process because oil and water do not mix. One way to mix oil and water is to make finely dispersed oil particles in water, which is referred to as an oil-in-water emulsion. For a long time, low-energy blenders/mixers have been used to produce these oil-in-water emulsions, typically resulting in 1 μm to 10 μm particle sizes. Such emulsions are blended into many food applications. These oil-in-water emulsions are suitable for many macro-nutrient (e.g., fat, protein, carbohydrate) delivery applications, such as, vegetable oils and fats that may or may not include flavored ingredients, such as, vanilla or chocolate.

If there is a need to add an oil-soluble substance of importance (e.g., a nutrient or drug) into a food application, an oil-in-water emulsion is a strategic route to do so effectively, provided the conditions for delivery are optimal. Typically, only a small amount of a nutrient or drug is added into the oil carrier before the oil-in-water emulsion is made. This may be referred to as micro-nutrient (e.g., vitamins, carotenoids, omega-3s, antioxidants, polyphenols) delivery.

In summary, to deliver a macro-nutrient oil or fat via the oil-in-water emulsion route, use of a low-energy mixer to produce particle sizes of 1 μm to 10 μm is suitable. However, to deliver a micro-nutrient (substance dissolved in the oil or fat) via the oil-in-water emulsion route using a low-energy mixer to produce 1 μm to 10 μm particles is not suitable. It is necessary to deliver these micro-nutrients wherein the particle sizes are less than 600 nm, sometimes referred to as sub-micron sizes. A high-energy homogenizer allows the oil-in-water emulsions to produce nanoparticles of typically 100 nm to 600 nm. That way, small amounts of nutrients (micro-nutrients) are delivered using the oil/fat as a carrier prior to blending with water, and therefore, making and sustaining the micro-nutrients in nanoparticles has numerous advantages, which is the subject of the disclosed compositions and methods.

Smaller nanoparticle sizes make more stable emulsions. Bigger microparticle sizes tend to clump (e.g., agglomerate and aggregate), causing the particles to break up and return to the two immiscible oil/fat and water layers. The much larger surface area (by as much as 100 to 10,000 times) produced by the nanoparticles (over the macroparticles) increases the chance of these small amounts of nutrients to be absorbed in the gut, which is known as bio-accessibility. Therefore, one end result of producing nanoparticles is bioavailability—what mammals and humans optimally receive in their internal system when they ingest these micro-nutrients via oil-in-water nanoemulsions. Using small amounts of nutrients minimizes flavor/taste alteration and reduces the use of excipients, hence increasing product safety and decreasing undesirable color changes.

The intention of the disclosed compositions and methods is to produce oil-in-water nanoemulsions that entrain oil-soluble nutrients. As a case in point, a plant-based vitamin E tocotrienol was added to an oil, put through a high-energy homogenizer with water, and nanoparticles of an oil-in-water emulsion were thus produced.

To be sustainably useful, it is further necessary to show that such manufactured nanoparticles are stable. The nanoparticles have electrostatic charges that are measured by those knowledgeable in the art to gauge its stability. These electrostatic charges are measured in millivolts and as zeta potentials. The zeta potential (measured in millivolts [my]) is a measurement of electrostatic forces of the generated nanoparticles. It is highly desirable for the nanoparticle potentials to repel each other to remain stable. When the nanoparticle potential is less than −30 mV, particles will repel each other and be stable. Similarly, when the nanoparticle potential is more than +30 mV, particles will repel each other and remain stable. A much higher negative (than −30 mV) or a much higher positive (than +30 mV) zeta potential shows much higher repulsive forces and implies even lower possibility for nanoparticles to aggregate, further implying higher stability. However, when the nanoparticle potential is between −30 mV and +30 mV, the particles are not strong enough to repel, and hence clump together to form larger aggregate particles, destabilizing the emulsion. Therefore, the disclosed compositions and methods produce nanoparticles that are stable.

Further, such stable nanoparticles must also remain stable when formulated in finished food or beverage formulations. An example of a failed nanoparticle delivery system means that the oil-in-water emulsion would break up, separate out, and the oil would float on top. As a case in point, stable nanoparticle tocotrienol emulsions were added to different beverages. They remained dispersed in the beverage under defined conditions (of pH, temperature, and duration) without taste/color difference or phase separation.

Another aspect of the disclosed compositions and methods is to replace the ubiquitous usage of synthetic products (petrochemically-derived chemicals) for emulsifiers and co-solvent to reduce viscosity of lipid and aqueous mixtures. It is the intention of the disclosed compositions and methods to replace all synthetic products with natural products (botanically-derived chemicals). Use of synthetic products may raise safety concerns, and the amount of synthetic products needed for successful emulsification may be high. The disclosed compositions and methods allow the use of lower amounts of botanically-derived emulsifiers, such as, saponins of quillaja and yucca. While synthetic emulsifiers increase bioavailability of nutrients, their use is self-limiting. In a case in point, synthetic emulsifiers (Cremophor and Labrasol) were added to annatto tocotrienol and nanoparticles were thus produced. It was shown that these synthetic emulsifiers inhibited tocotrienol absorption on average by 3.0 times to 5.0 times, a remarkable drop for emulsifier excipients added to enhance bioavailability. The study was dose-dependent, meaning as the amount of emulsifiers increased, the amount of tocotrienol absorbed by cells decreased. The use of natural emulsifiers will help obviate this problem. Further, the quantity of a natural emulsifier needed for an effective emulsification is much less than the quantity of a synthetic emulsifier. Furthermore, in the case of a synthetic emulsifier, 211 nm particles were produced, but the zeta potential attained was −25 mV, which is just above the −30 mV (or just outside the stable range) needed to be classified as a stable emulsion. The fact that the usage of synthetic emulsifiers was self-inhibiting and hence self-limiting in utility remained a major problem for the industry. This is not the case with natural emulsifiers.

Furthermore, in another aspect, the disclosed compositions and methods use co-solvent viscosity reducers that are botanically derived, such as, alcohols and terpenoids. These are used to minimize viscosity, and thereby allow the particle size to be the smallest possible and within the nanoparticle ranges.

### EMBODIMENTS

In one embodiment, there is a lipid solution and an aqueous solution. In another embodiment, the lipid solution is more than 10% and the aqueous solution is less than 90%. In another embodiment, the lipid solution is more than 25% and the aqueous solution is less than 75%. In one embodiment, the lipid solution is more than 50% and the aqueous solution is less than 50%.

In one embodiment, a co-solvent is added to the lipid solution. In another embodiment, a co-solvent is added to the lipid solution to reduce the viscosity of a liquid nutrient ingredient. In another embodiment, the co-solvent is a natural product. In another embodiment, the amount of the natural product co-solvent is a minimum to produce particle sizes of 50 nm-600 nm. In another embodiment, the amount of the natural product co-solvent is a minimum to produce particle sizes of 100 nm-400 nm. In another embodiment, the amount of the natural product co-solvent is a minimum to produce particle sizes of 100 nm-200 nm.

In one embodiment, the amount of the natural product co-solvent is a minimum to minimize the dilution of an active ingredient. In another embodiment, the amount of the co-solvent is 50% and the lipid nutrient or drug is 50%. In another embodiment, the amount of the co-solvent is 40% and the lipid nutrient or drug is 60%. In another embodiment, the amount of the co-solvent is 30% and the lipid nutrient or drug is 70%. In another embodiment, the amount of the co-solvent is 20% and the lipid nutrient or drug is 80%. In another embodiment, the amount of the co-solvent is 10% and the lipid nutrient or drug is 90%. In another embodiment, the lipid nutrient or drug is 100%.

In one embodiment, the natural product co-solvent is a naturally occurring terpenoid or alcohol.

In one embodiment, the terpenoid is limonene, farnesol or geranylgeraniol.

In one embodiment, the alcohol is ethanol or glycerol.

In one embodiment, a natural surfactant is added to the aqueous solution. In another embodiment, the natural surfactant is a saponin. In another embodiment, the saponin is quillaja, yucca or soy.

In one embodiment, a minimum amount of a saponin is used to attain a stable emulsion.

In one embodiment, the amount of the surfactant 20% and the aqueous solution is 80%. In another embodiment, the amount of the surfactant 10% and the aqueous solution is 90%. In another embodiment, the amount of the surfactant 5% and the aqueous solution is 95%. In another embodiment, the amount of the surfactant 1% and the aqueous solution is 99%. In another embodiment, the amount of the surfactant 0.5% and the aqueous solution is 99.5%.

In one embodiment, the lipid solution and the aqueous solution are blended and passed through a high-pressure homogenizer. In another embodiment, the blended lipid/aqueous solution is passed through the high-pressure homogenizer one to ten times. In another embodiment, the blended lipid/aqueous solution is passed through the high-pressure homogenizer two to six times. In another embodiment, the blended lipid/aqueous solution is passed through the high-pressure homogenizer two to four times.

In one embodiment, the repeated passes through the high-pressure homogenizer ensures a consistent form of nanoparticles.

In one embodiment, a zeta potential is measured after the repeated passes through the high-pressure homogenizer.

In one embodiment, a zeta potential is less than −30 mV or more than +30 mV. In one embodiment, a stable emulsion has a zeta potential less than −30 mV or more than +30 mV.

In one embodiment, a bioactive ingredient is stable in a nanoemulsion.

In one embodiment, blending and high-pressure homogenization does not oxidize the bioactive ingredient in the nanoemulsion.

In one embodiment, an inert gas is flushed through a headspace of an agitation vessel prior to high-pressure homogenization. In another embodiment, the inert gas is nitrogen or helium.

In one embodiment, the amount of the bioactive ingredient recovered in the nanoemulsion is from 90% to 100%. In another embodiment, the amount of the bioactive ingredient recovered in the nanoemulsion is from 90% to 95%.

In one embodiment, the stable nanoemulsion is used in food or beverage applications.

In one embodiment, an oil-in-water nanoemulsion is used in food or beverage applications.

In one embodiment, pH of the beverage is from 3.0 to 7.0 without degradation of the nanoemulsion.

In one embodiment, clarity of the beverage is clear, semi-clear or opaque without degradation of the nanoemulsion.

In one embodiment, the beverage is stored for a duration of 0 to 4 weeks without degradation of the nanoemulsion.

In one embodiment, a dispersed nanoemulsion of a tocotrienol from annatto seed is stable in a beverage of with a pH from 3.0 to 7.0 and a clarity of clear, semi-clear or opaque.

In one embodiment, a beverage is stored at a temperature from 20° C. to −20° C. without degradation of the nanoemulsion. In another embodiment, a beverage is stored at a temperature from 2° C. to −20° C. without degradation of the nanoemulsion. In another embodiment, a beverage is stored at a temperature from 2° C. to 7° C. without degradation of the nanoemulsion.

In one embodiment, a beverage is stored for a duration of 0 to 4 weeks and at a temperature from 20° C. to −20° C. and without degradation of the nanoemulsion. In another embodiment, a beverage is stored for a duration of 0 to 3 weeks and at a temperature from 20° C. to −20° C. and without degradation of the nanoemulsion. In another embodiment, a beverage is stored for a duration of 0 to 2 weeks and at a temperature from 20° C. to −20° C. and without degradation of the nanoemulsion.

In one embodiment, the nanoemulsion does not need to be color-masked or taste-masked.

In one embodiment, the amount of tocotrienol in a beverage is from 8% (v/w) to 17% (v/w). In another embodiment, the amount of tocotrienol in a beverage is from 33% (v/w) to 67% (v/w).

In one embodiment, the amount of tocotrienol in a beverage is from 8% (v/w) to 67% (v/w) without a change in taste or color of the beverage.

In one embodiment, the amount of the bioactive ingredient in a beverage is from 2% (v/w) to 84% (v/w). In another embodiment, the amount of the bioactive ingredient in a beverage is from 4% (v/w) to 42% (v/w). In another embodiment, the amount of the bioactive ingredient in a beverage is from 8% (v/w) to 21% (v/w).

In one embodiment, the bioactive ingredient is a lipid-soluble nutrient or a drug.

In one embodiment, the lipid-soluble nutrient is a vitamin E (tocotrienol or tocopherol), CoQ10 (ubiquinol or ubiquinone), curcuma terpenoids (xanthorrhizol, tumerones, curcumenes or curcumins), symmetrical carotenoids (astaxanthin, zeaxanthin, lycopene or beta-carotene), omega-3s (DHA or EPA), phenolics (policosanols, resveratrol, EGCG or quercetin), other lipid-soluble vitamins (A, D or K), and lipid-soluble pharmaceuticals.

Additional embodiments are described in the following paragraphs.

Paragraph 1. A method of making a lipid-soluble ingredient nanoemulsion comprising the steps of: a) mixing an active lipid-soluble ingredient and a lipid-soluble co-solvent to produce a lipid solution, b) mixing an emulsifier and an aqueous co-solvent to produce an aqueous solution, c) mixing the lipid solution and the aqueous solution together and homogenizing the two solutions under high pressure to generate emulsified particles of a lipid-soluble ingredient nanoemulsion.

Paragraph 2. The method of Paragraph 1, wherein the emulsified particle is from 50 nm to 600 nm in diameter.

Paragraph 3. The method of Paragraph 2, wherein the emulsified particle is from 100 nm to 400 nm in diameter.

Paragraph 4. The method of Paragraph 3, wherein the emulsified particle is from 100 nm to 200 nm in diameter.

Paragraph 5. The method of Paragraph 1, wherein a zeta potential is calculated for the emulsified particle and the zeta potential is less than −30 mV or more than +30 mV.

Paragraph 6. The method of Paragraph 1, wherein the aqueous solution further comprises a water-soluble natural surfactant.

Paragraph 7. The method of Paragraph 1, wherein the emulsifier is a saponin.

Paragraph 8. The method of Paragraph 7, wherein the saponin is selected from the group consisting of quillaja, yucca, and soy.

Paragraph 9. The method of Paragraph 1, wherein the active lipid-soluble ingredient is selected from the group consisting of vitamin E, CoQ10, curcuma terpenoid, symmetrical carotenoid, omega-3, phenolics, vitamin A, vitamin D, vitamin K, and lipid-soluble pharmaceuticals.

Paragraph 10. The method of Paragraph 9, wherein the vitamin E is selected from the group consisting of tocotrienol and tocopherol.

Paragraph 11. The method of Paragraph 9, wherein the tocotrienol is selected from the plant source consisting of annatto, palm, and rice.

Paragraph 12. The method of Paragraph 9, wherein the CoQ10 is selected from the group consisting of ubiquinol and ubiquinone.

Paragraph 13. The method of Paragraph 9, wherein the curcuma terpenoid is selected from the group consisting of xanthorrhizol, tumerones, curcumenes, and curcumins.

Paragraph 14. The method of Paragraph 9, wherein the symmetrical carotenoid is selected from the group consisting of astaxanthin, zeaxanthin, lycopene, and beta-carotene.

Paragraph 15. The method of Paragraph 9, wherein the omega-3 is selected from the group consisting of DHA and EPA.

Paragraph 16. The method of Paragraph 9, wherein the phenolics is selected from the group consisting of policosanol, resveratrol, EGCG, and quercetin.

Paragraph 17. The method of Paragraph 1, wherein the lipid-soluble co-solvent is a viscosity reducer.

Paragraph 18. The method of Paragraph 17, wherein the viscosity reducer is a natural terpenoid.

Paragraph 19. The method of Paragraph 17, wherein the natural terpenoid is selected from the group consisting of limonene, farnesol, geranylgeraniol and essential oil.

Paragraph 20. The method of Paragraph 1, wherein the aqueous co-solvent is a natural alcohol.

Paragraph 21. The method of Paragraph 20, wherein the natural alcohol is selected from the group consisting of ethanol and glycerol.

Paragraph 22. A method of making a nanoemulsion comprising the steps of: a) mixing an active lipid-soluble ingredient and a lipid-soluble co-solvent to produce a lipid solution, b) mixing an emulsifier and an aqueous co-solvent to produce an aqueous solution, c) mixing the lipid solution and the aqueous solution together and homogenizing the two solutions under high pressure to generate a nanoemulsion.

Paragraph 23. The method of Paragraph 22, wherein the nanoemulsion is added to a beverage.

Paragraph 24. The method of Paragraph 22, wherein the beverage has a clarity selected from the group consisting of clear, semi-clear and opaque.

Paragraph 25. The method of Paragraph 22, wherein the beverage has a pH from 3.0 to 7.0.

Paragraph 26. The method of Paragraph 22, wherein the beverage has a temperature from 20° C. to −20° C.

Paragraph 27. The method of Paragraph 22, wherein the aqueous solution further comprises a water-soluble natural surfactant.

Paragraph 28. The method of Paragraph 27, wherein the ratio of the surfactant to the aqueous solution is from 1:5 to 1:200.

Paragraph 29. The method of Paragraph 22, wherein the nanoemulsion is a liquid-liquid formulation.

Paragraph 30. The method of Paragraph 29, wherein the liquid-liquid formulation is a beverage.

Paragraph 31. The method of Paragraph 29, wherein the liquid-liquid formulation is an injectable.

Paragraph 32. The method of Paragraph 29, wherein the liquid-liquid formulation is an aerosol or aspirator product.

Paragraph 33. The method of Paragraph 29, wherein the liquid-liquid formulation is a douche.

Paragraph 34. The method of Paragraph 29, wherein the liquid-liquid formulation is a softgel.

Paragraph 35. The method of Paragraph 29, wherein the liquid-liquid formulation is an eye drop product.

Paragraph 36. The method of Paragraph 29, wherein the liquid-liquid formulation is an oral tincture product.

Paragraph 37. The method of Paragraph 29, wherein the liquid-liquid formulation is a skin care product.

Paragraph 38. The method of Paragraph 29, wherein the liquid-liquid formulation is a suppository.

Paragraph 39. The method of Paragraph 31, wherein the injectable is adapted for administering by the group consisting of subcutaneous, intramuscular and intravenous.

Paragraph 40. The method of Paragraph 22, wherein the nanoemulsion is added to a food product for mammals with a malabsorption condition.

Paragraph 41. The method of Paragraph 22, wherein the nanoemulsion has emulsified particles from 50 nm to 600 nm in diameter.

Paragraph 42. The method of Paragraph 41, wherein the emulsified particle is from 100 nm to 400 nm in diameter.

Paragraph 43. The method of Paragraph 42, wherein the emulsified particle is from 100 nm to 200 nm in diameter.

Paragraph 44. The method of Paragraph 41, wherein a zeta potential is calculated for the emulsified particle and the zeta potential is less than −30 mV or more than +30 mV.

Paragraph 45. The method of Paragraph 22, wherein the aqueous solution further comprises a water-soluble natural surfactant.

Paragraph 46. The method of Paragraph 22, wherein the emulsifier is a saponin.

Paragraph 47. The method of Paragraph 46, wherein the saponin is selected from the group consisting of quillaja, yucca, and soy.

Paragraph 48. The method of Paragraph 22, wherein the active lipid-soluble ingredient is selected from the group consisting of vitamin E, CoQ10, curcuma terpenoid, symmetrical carotenoid, omega-3, phenolics, vitamin A, vitamin D, vitamin K, and lipid-soluble pharmaceuticals.

Paragraph 49. The method of Paragraph 48, wherein the vitamin E is selected from the group consisting of tocotrienol and tocopherol.

Paragraph 50. The method of Paragraph 48, wherein the tocotrienol is selected from the plant source consisting of annatto, palm, and rice.

Paragraph 51. The method of Paragraph 48, wherein the CoQ10 is selected from the group consisting of ubiquinol and ubiquinone.

Paragraph 52. The method of Paragraph 48, wherein the curcuma terpenoid is selected from the group consisting of xanthorrhizol, tumerones, curcumenes, and curcumins.

Paragraph 53. The method of Paragraph 48, wherein the symmetrical carotenoid is selected from the group consisting of astaxanthin, zeaxanthin, lycopene, and beta-carotene.

Paragraph 54. The method of Paragraph 48, wherein the omega-3 is selected from the group consisting of DHA and EPA.

Paragraph 55. The method of Paragraph 48, wherein the phenolics is selected from the group consisting of policosanol, resveratrol, EGCG, and quercetin.

Paragraph 56. The method of Paragraph 22, wherein the lipid-soluble co-solvent is a viscosity reducer.

Paragraph 57. The method of Paragraph 56, wherein the viscosity reducer is a natural terpenoid.

Paragraph 58. The method of Paragraph 56, wherein the natural terpenoid is selected from the group consisting of limonene, farnesol, geranylgeraniol and essential oil.

Paragraph 59. The method of Paragraph 22, wherein the aqueous co-solvent is a natural alcohol.

Paragraph 60. The method of Paragraph 59, wherein the natural alcohol is selected from the group consisting of ethanol and glycerol.

Paragraph 61. The method of Paragraph 1, wherein the nanoemulsion is added to a beverage.

Paragraph 62. The method of Paragraph 1, wherein the beverage has a clarity selected from the group consisting of clear, semi-clear and opaque.

Paragraph 63. The method of Paragraph 1, wherein the beverage has a pH from 3.0 to 7.0.

Paragraph 64. The method of Paragraph 1, wherein the beverage has a temperature from 20° C. to −20° C.

Paragraph 65. The method of Paragraph 1, wherein the aqueous solution further comprises a water-soluble natural surfactant.

Paragraph 66. The method of Paragraph 65, wherein the ratio of the surfactant to the aqueous solution is from 1:5 to 1:200.

Paragraph 67. The method of Paragraph 1, wherein the nanoemulsion is a liquid-liquid formulation.

Paragraph 68. The method of Paragraph 67, wherein the liquid-liquid formulation is a beverage.

Paragraph 69. The method of Paragraph 67, wherein the liquid-liquid formulation is an injectable.

Paragraph 70. The method of Paragraph 67, wherein the liquid-liquid formulation is an aerosol or aspirator product.

Paragraph 71. The method of Paragraph 67, wherein the liquid-liquid formulation is a douche.

Paragraph 72. The method of Paragraph 67, wherein the liquid-liquid formulation is a softgel.

Paragraph 73. The method of Paragraph 67, wherein the liquid-liquid formulation is an eye drop product.

Paragraph 74. The method of Paragraph 67, wherein the liquid-liquid formulation is an oral tincture product.

Paragraph 75. The method of Paragraph 67, wherein the liquid-liquid formulation is a skin care product.

Paragraph 76. The method of Paragraph 67, wherein the liquid-liquid formulation is a suppository.

Paragraph 77. The method of Paragraph 69, wherein the injectable is adapted for administering by the group consisting of subcutaneous, intramuscular and intravenous.

Paragraph 78. The method of Paragraph 1, wherein the nanoemulsion is added to a food product for mammals with a malabsorption condition.

### EXAMPLES

### Example 1

Two separate solutions were made, a 10% lipid solution and a 90% aqueous solution. In the lipid solution, 5 g of vitamin E (70% tocotrienol from annatto seed) was added to 5 g of a co-solvent (limonene oil) to make the 10 g lipid solution. In the aqueous solution, 3.6 g of a natural tree-bark surfactant (quillaja saponins) was dissolved in pH 7.0 buffered water to make up 90 g of aqueous solution. These two solutions were first blended, and then put three times through a high-pressure homogenizer. A milky yellow emulsion was formed. The ratio of Surfactant to Solution was 1 to 28. Limonene was used to minimize viscosity. Zeta potential was measured using a light-scattering electrophoretic mobility instrument (Malvern Instruments).

The size of the nanoparticle and the stability of the emulsion were:

Particle Size: 115 nm

Zeta Potential: −64 mV

Tocotrienol (w/w): 3.5%

Visual Suspension: no creaming, no precipitation

### Example 2

Tocotrienol (vitamin E from annatto) was used as the bioactive component. The tocotrienol was extracted from the solution, before and after emulsion homogenization, and were measured by HPLC to test the stability of composition. Acceptable losses were observed through the emulsion process.

The results were as follows:

A] Concentrations of tocotrienol and limonene oil were 0.95 and 0.84 g/ml, respectively.
B] 5 g tocotrienol from annatto seed (5.26 ml)+5 g limonene oil (5.95 ml)=10 g (11.2 ml)
C] Tocotrienol (v/v) is (5.26/11.2)×7% [concentration of tocotrienol in the nanoemulsion]=3.29%
D] HPLC tocotrienol analysis=3.0%
E] % Recovery/Yield is (3.0/3.29)×100=91.2% (loss of <9%)

High-pressure homogenization causes severe agitation of the lipid and aqueous phases that may introduce air (˜20% oxygen) into the solution; which may oxidize a bioactive ingredient in the solution. This potential oxidation may cause unwanted degradation or reduce the bioactive ingredient.

This experiment showed that the high-pressure homogenization did not cause an undesirable degradation of the tocotrienol (bioactive ingredient) in the emulsion with a surfactant in the aqueous phase. HPLC analysis showed a recovery of more than 90%. A recovery of less than 80% (i.e., a loss of greater than 20%) of the active form of the bioactive ingredient would not be acceptable.

### Example 3

Beverages were used to test oil-in-water emulsions produced by the disclosed compositions and methods. An average of 10 mg-20 mg tocotrienol were mixed into 30 ml cups of beverages to measure the stability of the emulsions. Beverages included water, apple juice, orange juice, lemonade, milk, and chocolate milk.

Therefore, in a 240 ml serving, 80-160 mg of tocotrienol would be used in the beverage. The taste of the beverage was unchanged with or without the emulsified tocotrienol, and there was no phase separation.

### Example 4

Several beverages were chosen based on their acidity and clarity, and subjected to different storage conditions of temperature (room temperature, 25° C.; refrigeration, 5° C.; freezer, −15° C.) and duration (0 to 4 weeks). The experimental design is shown in Table 2.

TABLE 2

| | | | | Duration |
|---|---|---|---|---|
| Beverage | pH | Clarity | Temperature (° C.) | (weeks) |
| Water | 7.0 | Clear | 5 | 0-4 |
| Apple Juice | 3.4 | Clear | 5 | 0-3 |
| Orange Juice | 3.0 | Semi-Clear | 5 | 0-4 |
| Lemonade | 3.0 | Semi-Clear | −15 to 25 | 0-4 |
| Milk | 6.7 | Opaque | 5 | 0-2 |
| Chocolate Milk | 6.8 | Opaque | 5 | 0-2 |

### Example 5

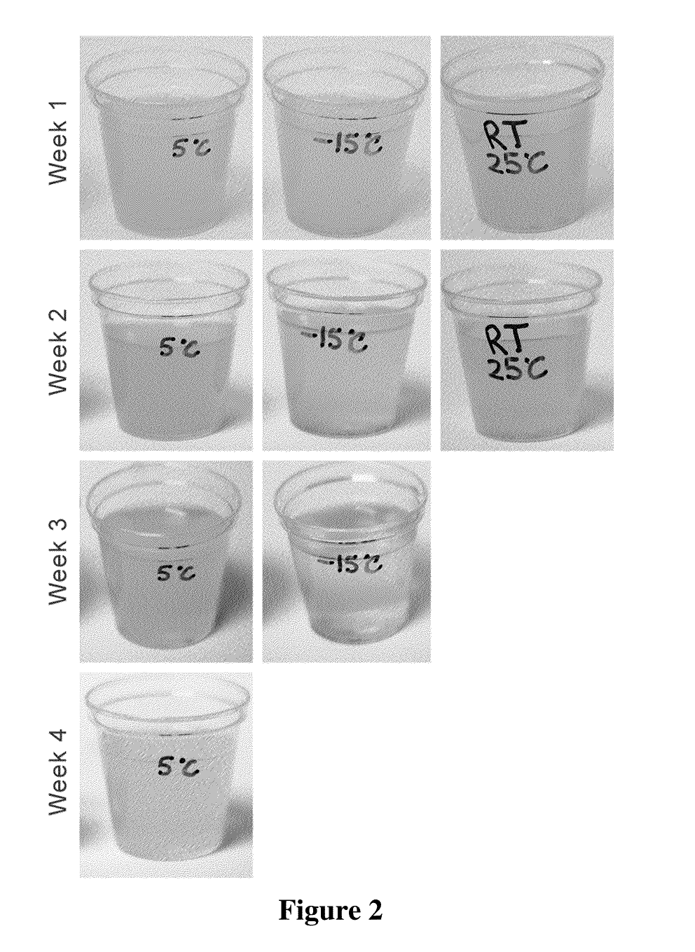

FIGS. 2-5 illustrate the relative stability of the emulsion with different acidity, temperature and duration of storage. No change in clarity was seen with clear solutions. Lack of creaming (floating matter) and precipitation (settling matter) in the various beverages was observed at temperatures from −15° C. to 25° C. and durations from 0 to 4 weeks.

FIG. 2 shows a primary emulsion in lemonade subjected to various temperatures to simulate storage conditions at room temperature (25° C.), refrigeration (5° C.), and freezing (−15° C.). Creaming was not observed at any of the temperature conditions. The emulsion was stable for just two weeks at 25° C. because fermentation was observed to begin on week 3. The emulsion was stable for three weeks at −15° C.; however, precipitation was observed by the 4^{th} freeze-thaw cycle on week 4. The emulsion was entirely stable for at least four weeks at 5° C. without phase separation.

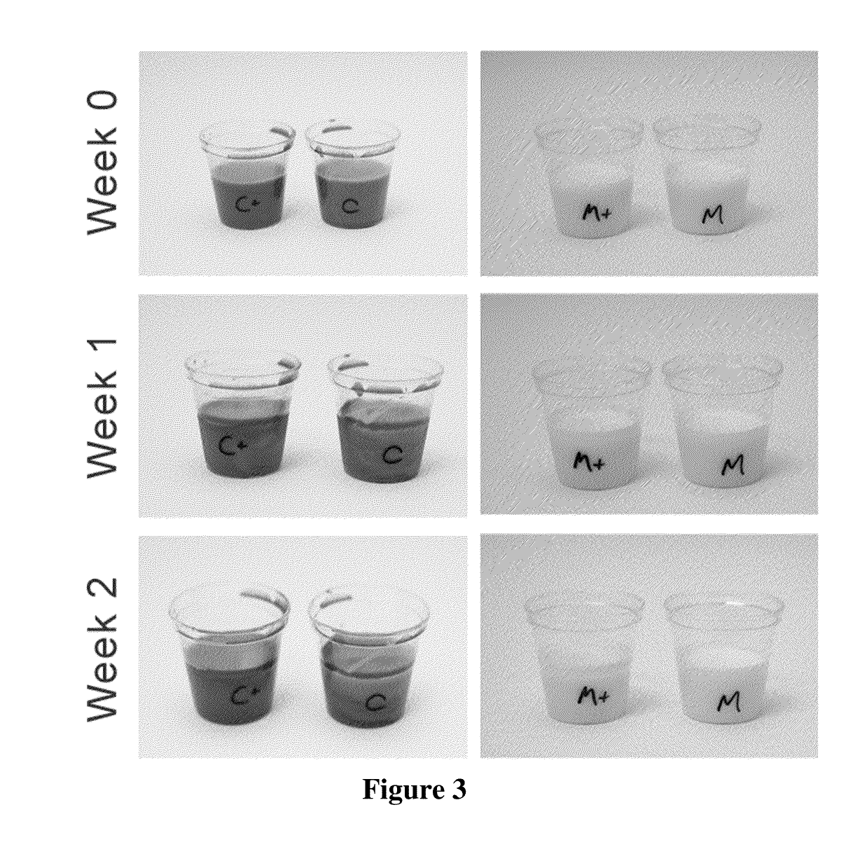

FIG. 3 shows a primary emulsion added into near neutral pH chocolate milk with (C+) and without (C) emulsified ingredients. Additionally, milk was tested with (M+) and without (M) emulsified ingredients. It was expected that these milk-based products would last for at least two weeks with refrigeration after opening the containers.

These products were stable for at least two weeks at 5° C. Creaming was observed on week 3. The taste of milk based products with or without emulsified ingredients was indistinguishable at the end of week 2 when tasted and there was no phase separation. Duration of stability is indicated in each figure.

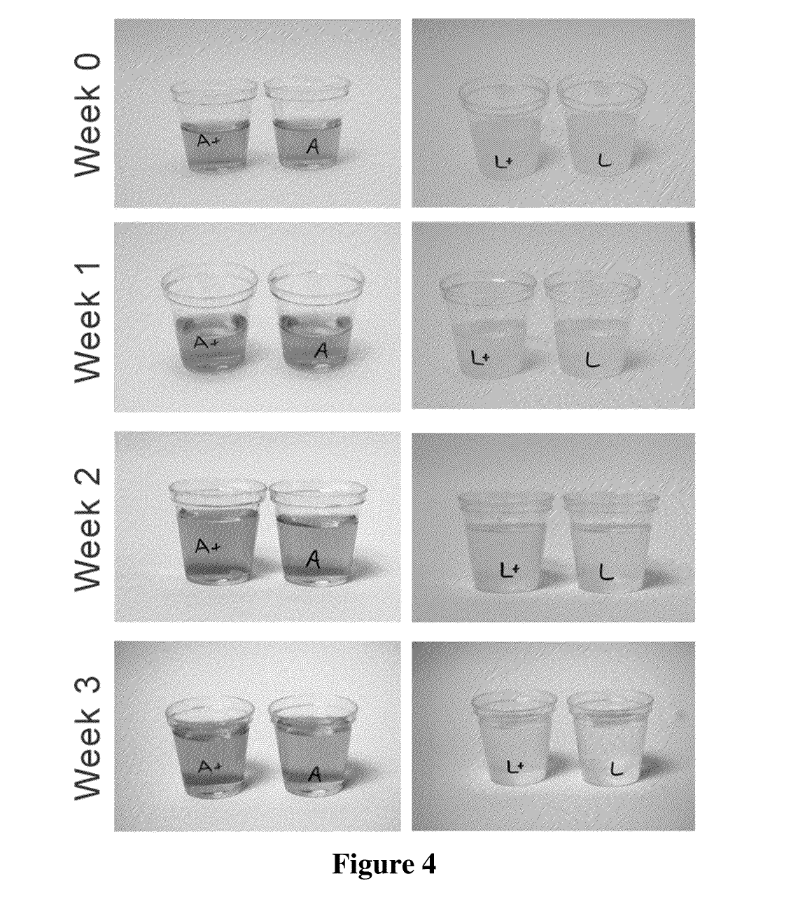

FIG. 4 shows a primary emulsion solution added to acidic (pH 3.4) apple juice with (A+) and without (A) emulsified ingredients. Lemonade was also similarly tested and labeled with (L+) and without (L) emulsified ingredients. These products were expected to last 3 weeks after opening the containers because of their acidic condition. The products were stable for at least three weeks at 5° C. Cloudiness appeared on week 4. The taste of these products with and without emulsified ingredients was indistinguishable at the end of week 3 when tasted and there was no phase separation.

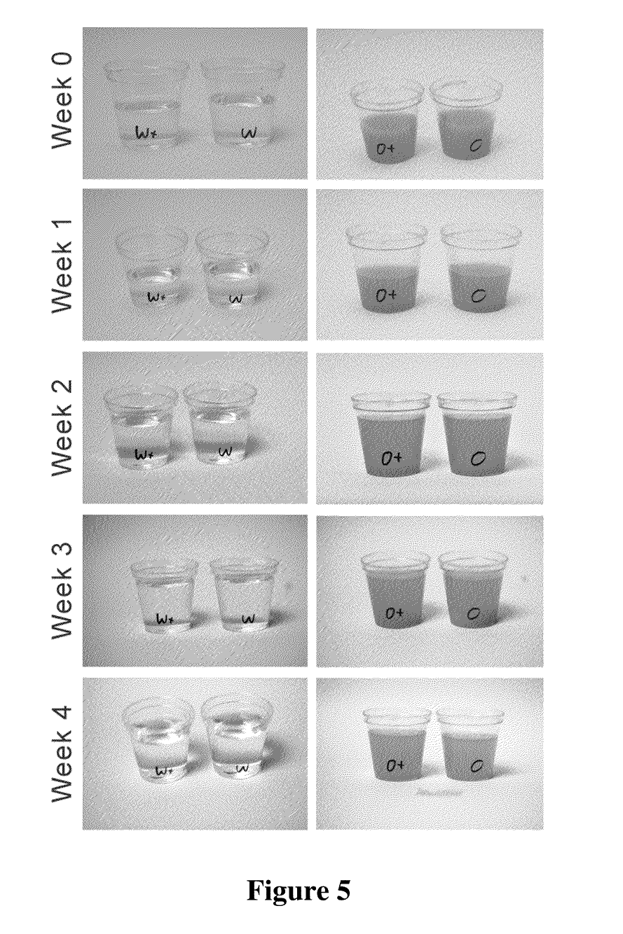

FIG. 5 shows a primary emulsion solution added to water (pH 7.0) and orange juice (pH 3.0). Water with (W+) and without (W) emulsion ingredients and orange juice with (O+) and without (O) emulsion ingredients were tested and labeled, accordingly. The clear water drink could represent spring water, purified water, mineral/vitamin water and flavored water. There were no changes (e.g., creaming, cloudiness, precipitation, color) and these two products were stable for at least four weeks at 5° C. The taste of these products with and without emulsified ingredients was indistinguishable at the end of week 4 when tasted and there was no phase separation.

### Example 6

Using the method in Example 1, 1 ml of the nanoemulsion (containing 8% of tocotrienol) in a 240 ml (clear) beverage will deliver 80 mg tocotrienol/serving. In a chocolate-milk or milk based drinks (opaque), 2 ml of the nanoemulsion (containing 8% of tocotrienol) will deliver 160 mg tocotrienol/serving. An antioxidant or juice drink (semi-clear) containing 0.5 ml of the nanoemulsion (containing 8% of tocotrienol) will deliver 40 mg tocotrienol/serving. A water or apple juice beverage (clear) containing 0.125 ml of the nanoemulsion (containing 8% of tocotrienol) will deliver 10 mg tocotrienol/serving. A food application containing 0.1 ml of the nanoemulsion (containing 8% of tocotrienol) will deliver 8 mg tocotrienol/serving. These will satisfy the applications in FDA GRAS-approved usage.

### Example 7

Using the method in Example 1, in a subcutaneous (SQ), intravenous (IV) or intramuscular (IM) injection application or oral application (for a person with mal-absorption syndrome), 50% Aqueous and 50% Lipid ratio will yield 40% tocotrienol, and 2 ml of 40% tocotrienol can deliver 800 mg tocotrienol/serving.

### Example 8

Using the method in Example 1, in another injectable application (e.g., SQ, IM, IV) or oral applications (for a person with mal-absorption syndrome), 30% Aqueous and 70% Lipid ratio will yield 56% tocotrienol, and 2 ml of 56% tocotrienol can deliver 1,120 mg tocotrienol/serving.

1. A method of making a lipid-soluble ingredient nanoemulsion comprising the steps of: a) mixing an active lipid-soluble ingredient and a lipid-soluble co-solvent to produce a lipid solution, b) mixing an emulsifier and an aqueous co-solvent to produce an aqueous solution, c) mixing the lipid solution and the aqueous solution together and homogenizing the two solutions under high pressure to generate emulsified particles of a lipid-soluble ingredient nanoemulsion.

2. The method of claim 1, wherein the emulsified particle is from 50 nm to 600 nm in diameter.

3. The method of claim 2, wherein the emulsified particle is from 100 nm to 400 nm in diameter.

4. The method of claim 1, wherein a zeta potential is calculated for the emulsified particle and the zeta potential is less than −30 mV or more than +30 mV.

5. The method of claim 1, wherein the aqueous solution further comprises a water-soluble natural surfactant.

6. The method of claim 1, wherein the emulsifier is a saponin.

7. The method of claim 6, wherein the saponin is selected from the group consisting of quillaja, yucca, and soy.

8. The method of claim 1, wherein the active lipid-soluble ingredient is selected from the group consisting of vitamin E, CoQ10, curcuma terpenoid, symmetrical carotenoid, omega-3, phenolics, vitamin A, vitamin D, vitamin K, and lipid-soluble pharmaceuticals.

9. The method of claim 8, wherein the vitamin E is selected from the group consisting of tocotrienol and tocopherol.

10. The method of claim 9, wherein the tocotrienol is selected from the plant source consisting of annatto, palm, and rice.

11. The method of claim 8, wherein the CoQ10 is selected from the group consisting of ubiquinol and ubiquinone.

12. The method of claim 8, wherein the curcuma terpenoid is selected from the group consisting of xanthorrhizol, tumerones, curcumenes, and curcumins.

13. The method of claim 8, wherein the symmetrical carotenoid is selected from the group consisting of astaxanthin, zeaxanthin, lycopene, and beta-carotene.

14. The method of claim 8, wherein the omega-3 is selected from the group consisting of DHA and EPA.

15. The method of claim 8, wherein the phenolics is selected from the group consisting of policosanol, resveratrol, EGCG, and quercetin.

16. The method of claim 1, wherein the lipid-soluble co-solvent is a viscosity reducer.

17. The method of claim 16, wherein the viscosity reducer is a natural terpenoid.

18. The method of claim 16, wherein the natural terpenoid is selected from the group consisting of limonene, farnesol, geranylgeraniol and essential oil.

19. The method of claim 1, wherein the aqueous co-solvent is a natural alcohol.

20. The method of claim 19, wherein the natural alcohol is selected from the group consisting of ethanol and glycerol.
